(19) United States
(12) Patent Application Publication
(10) Pub. No.: US 2023/0080299 A1
(43) Pub. Date: Mar. 16, 2023

(54) TOOTH-WHITENING GEL COMPRISING MODIFIED NIOBIUM COMPOUNDS, METHOD AND USE

(71) Applicants: UNIVERSIDADE FEDERAL DE MINAS GERAIS, Belo Horizonte (BR); ODONTO TECH PESQUISA E INOVACAO LTDA, Savassi (BR)

(72) Inventors: Luiz Carlos ALVES DE OLIVEIRA, Pampulha (BR); Jadson Claudio BELCHIOR, Pampulha (BR); Luis Fernando MORGAN DOS SANTOS ALVES, Pampulha (BR); Ana Pacheli HEITMANN RODRIGUES, Pampulha (BR); Fernando Antonio MAUAD DE ABREU, Pampulha (BR); Cinthia DE CASTRO OLIVEIRA, Pampulha (BR); Francine BENETTI FARIA, Pampulha (BR); Poliane CHAGAS, Pampulha (BR); Camila DE SOUSA CANESCHI, Pampulha (BR)

(21) Appl. No.: 18/050,168

(22) Filed: Oct. 27, 2022

Related U.S. Application Data
(63) Continuation-in-part of application No. PCT/IB2021/056144, filed on Jul. 8, 2021.

(30) Foreign Application Priority Data
Jul. 28, 2020 (BR) 1020200153676
Nov. 25, 2021 (BR) 1320210238053

(51) Int. Cl. A61K 8/19; A61K 8/22; A61K 8/46; A61K 8/73; A61Q 11/00

(52) U.S. Cl. CPC A61K 8/19; A61K 8/22; A61K 8/463; A61K 8/731; A61Q 11/00; A61K 2800/48

(57) ABSTRACT

A teeth whitening based on the generation of active oxygen in the form of oxygen radical species in situ and method for producing the whitening gel which has modified niobium compounds and its activity maximized if applied with hydrogen peroxide in low concentrations, with stabilization by a thickener. The whitening gel can be used without light treatment and with greater efficiency than commercial products and with a significant potential to reduce side effects. The whitening toothpaste and gels including modified niobium compounds associated with cations, the mechanisms of whitening action comes from electronic excitation, from the incidence of radiation on niobium compounds with cations, due to the action of the functional chemical groups present in the nanoparticles, even in the absence of light. In addition, the compounds can act as chemical whiteners, leading to a decrease in caries bacteria due to the oxidant functional groups in the nanoparticles.

### CROSS-REFERENCE TO RELATED APPLICATIONS

This application is a bypass continuation-in-part of PCT Application Serial No. PCT/IB2021/056144, filed Jul. 8, 2021, which in turn claims the benefit of priority from Brazilian Patent Application No. 1020200153676, filed Jul. 28, 2020, and claims the benefit of priority from Brazilian Patent Application No. 1320210238053, filed Nov. 25, 2021, the contents of each of which are incorporated herein by reference.

### BACKGROUND OF THE INVENTION

The present technology relates to products applied for teeth whitening based on the generation of active oxygen in the form of oxygen radical species in situ. The present technology also describes its method of production. The whitening gel comprises modified niobium compounds and its activity is maximized mainly if it is applied with hydrogen peroxide in low concentrations. The whitening gel can be used in teeth whitening without the need for additional light to the treatment, which allows its use in teeth whitening with greater efficiency than commercial products and with a significant potential to reduce side effects (hypersensitivity and inflammation). The present technology also relates to whitening toothpaste and gels comprising modified niobium compounds associated with cations, wherein the mechanisms of whitening action come from electronic excitation, from the incidence of radiation on niobium compounds with cations, which may be due to the action of the functional chemical groups present in the nanoparticles, even in the absence of light. In addition, when dispersed in toothpaste, the compounds can act as chemical whiteners, in addition to leading to a decrease in caries bacteria due to the oxidant functional groups in the nanoparticles.

In recent years, there has been a significant increase in the demand for teeth whitening in aesthetic clinics and dental offices. However, in commercial products such as whitening or lightening gels, high concentrations (6 to 35%) of hydrogen peroxide (H₂O₂) are used in their formulations. Exposure of teeth to peroxide can cause side effects such as sensitivity, damage to the pulp, tooth enamel and periodontal tissue. In the state of the art, there are several studies highlighting the importance of minimizing these adverse effects using materials based on inorganic compounds (Application of Titanium Dioxide Nanotubes to Tooth Whitening, Nano Biomedicine, Volume 6, Issue 2, 12 Dec. 2014, Pages 63-72) (Synthesis of metal ion-histidine complex functionalized mesoporous silica nanocatalysts for enhanced light free tooth bleaching, Acta Biomaterialia, Volume 7, Issue 5, 7 Jan. 2011, Pages 2276-2284)

Tooth whitening is a procedure routinely used in dental offices to improve dental aesthetics. The conventional whitening procedure consists of applying a gel based on hydrogen peroxides or carbamide for better stability. However, whitening from the use of peroxides may be associated with adverse effects, such as structural changes, demineralization of the tooth surface and subsurface, reductions in surface microhardness, and increased surface roughness of enamel and dentin (Chen, Ying-Hui et al. Journal of Dentistry, v. 95, 103318, 2020).

Although the use of tooth whiteners with low concentrations of peroxide represents an advantage as it is less aggressive to exposed tissues, the need to produce tooth whiteners totally free of these components in their formulations is evident.

The scientific literature reports several studies highlighting the importance of minimizing the adverse effects of the use of peroxides or replacing peroxides with other whitening components or by the process of incidence of radiation on photobleaching compounds.

The use of photosensitive materials acting in tooth whitening processes is widely reported in the state of the art, as discussed below.

In the state of the art, it is also possible to find several studies highlighting the use of niobium compounds for use in oxidation reactions, including organic pollutants present in industrial effluents (Photocatalytic degradation of hazardous Ponceu-S dye from industrial wastewater using nanosized niobium pentoxide with carbon, Desalination, Volume 269, Issue 1-3, 4 Nov. 2010, Pages 276-283) (Amphiphilic niobium oxyhydroxide as a hybrid catalyst for sulfur removal from fuel in a biphasic system, Applied Catalysis B: Environmental, Volume 147, 6 Aug. 2013, Pages 43-48).

The use of reactive materials acting in teeth whitening is reported in the literature in different documents. Document US20040180008, from 2004, entitled “Dental bleaching agent kit and the method for bleaching teeth” and US20060222604, from 2002, with the following title “Method for bleaching teeth and bleaching agent for teeth” report the combined use of titanium compounds and visible light based on the photocatalysis reaction. In addition, the importance of using peroxides to ensure teeth whitening is emphasized.

The invention related to U.S. Pat. No. 5,645,428, from 1995, entitled “Method for whitening teeth” reports the efficiency of teeth whitening using a mixture of peroxides with different catalysts exposed to argon laser radiation and also with carbon dioxide laser. This method employs other materials including buffers, stabilizers, desensitizers and thickeners.

In U.S. Pat. No. 5,032,178, from 1990, entitled “Dental composition system and method for bleaching teeth”, materials from the manganese sulfate and iron sulfate classes are described, which were used together with other products, including hydrogen peroxide and presence of visible radiation in teeth whitening.

Document BR102013027175-6, filed in 2013, entitled “Teeth whitening accelerator”, relates to a mixture of ferric nitrate and cupric nitrate together with hydrogen peroxide promoting efficient whitening.

Natural catalysts such as enzymes of the peroxidases family such as catalase are described in the document BR102014010685-5, filed in 2014, entitled “Teeth whitening maximizer”, having a rapid decomposition of hydrogen peroxide or carbamide peroxide with efficient action of teeth whitening.

Document PI0801862-6, from 2008, entitled “Teeth whitening gel with micro or nano solid particles that absorb energy and is thermally conductive”, relates to conductive inorganic, ceramic or organic micro or nanoparticles that are incorporated into the peroxide-based whitening gel, which, in addition to providing greater efficiency in teeth whitening, minimizes side effects, such as hypersensitivity.

The use of materials based on niobium compounds, highlighting the unprecedented use of the oxalate salt (which could be niobium pentoxide, niobic acid and oxyhydroxide) in the activation of hydrogen peroxide or organic peroxides, proposed in the present technology, applied to teeth whitening is unprecedented, mainly with the generation of radical oxygen in situ due to the reaction of niobium with H₂O₂, as well as the stabilization of oxygen species with commercial carbopol. Another novelty of the present invention consists in the fact that the added hydrogen peroxide is decomposed by the reaction with niobium species, generating active oxygen species, decreasing the presence of free peroxide, causing the sensitivity to be eliminated. In this way, it is possible to reduce the side effects of hypersensitivity during treatment, in addition to allowing the production of a material with low cost, which can facilitate its commercialization.

The oxygen species generated in the dissociation of H₂O₂ have an oxidizing action capable of breaking the chemical bonds of the molecular chains of the chromophore groups (substances that give teeth color). Low concentrations of peroxide are sufficient for this effect (approximately 2% by mass) in the product presented due to three technological innovations: (i) the niobium compound acts efficiently and quickly in its activation to generate reactive radical species; (ii) decrease in free peroxide, decreasing sensitivity during bleaching (iii) carbopol stabilizes radicals preventing their decomposition, which enhances teeth whitening.

In addition, with the use of this gel in teeth whitening there is no need to use radiation for the decomposition of the peroxide, significant change in pH and with that it is possible to reduce the treatment time in the office or at home. These features provide the product generated in the present technology with great commercial potential.

The present technology relates to the development of material using sources of niobium as raw material to obtain the whitening nanoparticles. The use of niobium compounds in the production of niobium-containing teeth whitening gel, as proposed in the present technology, is not reported in the scientific literature and may promote another commercial application for this important chemical element, which is currently used mainly in the metallurgical industry.

The present technology also relates to whitening toothpaste and gels comprising modified niobium compounds associated with cations, method of obtaining and use thereof. The niobium compounds give the product the ability to act in tooth whitening with visible radiation, the radiation can be natural or artificial, without the need to use free peroxides. The mechanisms of whitening action come from electronic excitation, from the incidence of radiation on niobium compounds with cations, which may be due to the action of the functional chemical groups present in the nanoparticles, even in the absence of light. In addition, when dispersed in a toothpaste, the compounds can act as chemical whiteners, in addition to leading to a decrease in caries bacteria due to the oxidant functional groups in the nanoparticles.

Patent document US20040191729A1, whose priority date is Nov. 29, 2001, entitled “Dental phototherapy methods and compositions”, reports the application of a dental bleaching and antimicrobial compound based on a non-toxic chromophore that is activated by radiation in the visible, ultraviolet and infrared regions. The chromophore can act as a dental bleaching and/or antimicrobial agent and can also be applied to treat oral diseases such as caries and periodontitis.

The patent document JP2004292429A, the priority date of which is Mar. 10, 2003, entitled “Bleaching agent set for teeth and method for bleaching teeth”, describes a tooth whitener with photobleaching action. The whitener described is composed of titanium oxide powder, titanium oxynitride and/or titanium oxide doped with nitrogen dispersed in an organic solvent and must also comprise a thickener. The formation of hydrogen peroxide from the contact of the whitener with water is described, so that the bleaching activity of the product is associated with the presence of hydrogen peroxide combined with the photocatalytic activity of the titanium compounds.

Patent document BR1020200153676, entitled “TOOTH WHITENING GEL COMPRISING MODIFIED NIOBIUM COMPOUNDS, METHOD AND USE”, the priority date of which is 07/28/2020, relates to a whitening gel containing niobium compounds modified by leaching with peroxides dispersed in a polymer matrix. The gel must be applied together with hydrogen peroxide to present the whitening activity. It is worth mentioning that the use of free peroxides in the whitening process induces sensitization of the teeth due to demineralization and damage to tooth enamel and dentin. The association of cation-modified niobium compounds or the photosensitive activity of the material is not mentioned.

In the state of the art, the use of niobium nanoparticles modified with cations, the photosensitive niobates, was not found to promote tooth whitening with the action of visible light, from a product totally free of free peroxides.

The present technology relates to tooth whiteners comprising photosensitive niobium compounds modified with cations. Its advantages compared to the state of the art are the absence of free peroxides in the whitening process and the use of radiation in the visible region. The addition of cations to niobium compounds, in adequate concentrations, allows the precipitation of niobium compounds associated with cations, which have functional groups that can act as whiteners even in the absence of radiation and are photosensitive. Thus, from the incidence of radiation, the new cation-associated niobium compounds have their activity to degrade the molecules that darken the teeth increased. In this way, the activity of niobium compounds with cations results in dispensing the use of peroxides during the whitening process. The absence of free peroxides in the product formulation mainly results in the protection of tooth tissues. It is important to emphasize that the radiation used is of high wavelength, reducing operating costs and avoiding damage to the client's health, due to its low energy. Finally, the use of niobates renders these products novel by employing this element outside of metallurgy, where it is mostly used. Brazil has the largest reserves and is the world's largest producer of niobium, thus, its application in several areas is interesting and economically promising.

### BRIEF DESCRIPTION OF DRAWINGS

FIG. 1 shows an applicator syringe containing teeth whitening gel.

FIG. 2 presents parameters of brightness, intensity of color/luminosity and shade obtained through the VITA 3D MASTER scale using different niobium compounds.

FIG. 3 shows photographs of bovine teeth before and after treatment with materials G1 (A) and G3 (B).

FIG. 4 shows SEM images of the teeth of rats submitted to teeth whitening with whitening gel G1 (A), commercial G3 (B) and the control group (C) with 200× magnification (top pictures) and 500× (lower pictures).

FIG. 5 presents representative images of sections stained with hematoxylin and eosin showing the coronal pulp 2 days after bleaching. The panels represent the groups treated with the whitening gel, the commercial product (G3) for 45 minutes and the control group, with 40× and 100× magnification.

FIG. 6 shows the whitening process with photosensitive niobates in bovine teeth. In a), the application of niobates on a bovine tooth; in b), the incidence of radiation from the visible region on teeth with niobates; in c), the tooth before whitening; and in d), the tooth after whitening.

FIG. 7 presents a graph in which the whitening effect of a commercial product containing 35% of hydrogen peroxide and methylene niobate is quantitatively compared, using DE.

FIG. 8 shows a graph in which the whitening effect of a commercial product A containing 2% hydrogen peroxide, a commercial toothpaste with 2% hydrogen peroxide; B, commercial niobium oxide toothpaste; C, toothpaste with solidified niobate without the addition of cation in the ratio of 3% m/m of niobate; D, toothpaste with niobate solidified with Ca2⁺ in the ratio of 3% m/m of calcium niobate; E, toothpaste with niobate solidified with Fe3+ in the ratio of 3% of iron (III) niobate; F, toothpaste with niobates obtained from commercial niobium phosphate in the ratio of 1% m/m of niobates; and G, toothpaste with niobates obtained from commercial niobium phosphate in the ratio of 1% m/m of niobates.

### DETAILED DESCRIPTION OF THE INVENTION

The present technology relates to products applied for teeth whitening based on the generation of active oxygen in the form of oxygen radical species in situ. The present technology also describes its method of production. The whitening gel comprises modified niobium compounds and its activity is maximized mainly if it is applied with hydrogen peroxide in low concentrations, from 1 to 10% (preferably 3% by mass), with stabilization by a thickener, preferably carbopol. The whitening gel can be used in teeth whitening without the need for additional light to the treatment, which allows its use in teeth whitening with greater efficiency than commercial products and with a significant potential to reduce side effects (hypersensitivity and inflammation). The present technology also relates to whitening toothpaste and gels comprising modified niobium compounds associated with cations, wherein the mechanisms of whitening action come from electronic excitation, from the incidence of radiation on niobium compounds with cations, which may be due to the action of the functional chemical groups present in the nanoparticles, even in the absence of light. In addition, when dispersed in a toothpaste, the compounds can act as chemical whiteners, in addition to leading to a decrease in caries bacteria due to the oxidant functional groups in the nanoparticles.

The teeth whitening gel comprises niobium compounds modified by the previous reaction with peroxides and a thickener, in the ratio between 1 and 50% by mass of the niobium compound in relation to the thickener.

The thickener used can be selected from the group comprising natrosol, xanthan gum, hydroxymethylcellulose, carbomer, carbopol or combinations thereof, or toothpaste or pure glycerin.

In step “a” the niobium compounds are selected from the group comprising niobium phosphates, niobium oxides, acetates, chlorides, niobium filter cake, niobia, or the oxalate anion ([Nb(O) (C₂O₄)₃]³⁻).

The peroxides used are selected from the group comprising methyl ethyl ketone peroxide, benzoyl peroxide, carbamide peroxide, or hydrogen peroxide, with a purity between 30 and 70%, in concentrations between 1, 0 and 10.0% m/m of peroxide in relation to the total mass of the gel (thickener and niobium compound).

The teeth whitening gel preparation process comprises the following steps:
a. Modifying the niobium compounds, generating surface oxidizing oxygen groups by reacting with hydrogen peroxide of purity between 30 and 70%, using peroxide concentrations between 1.0 and 10.0% m/m in relation to the total mass (thickener and compound of niobium);
b. Adding the thickener in the ratio of 1 to 10% by mass of the modified niobium compound obtained in step “a” in relation to the thickener;
c. Mixing the composition obtained in “b” under gentle agitation between 50 and 1000 rpm for a time interval between 10 and 60 min, at room temperature.

In step “a” the niobium compounds are selected from the group comprising niobium phosphates, niobium oxides, acetates, chlorides, niobium filter cake, niobia, or the oxalate anion ([Nb(O) (C₂O₄)₃]³⁻).

In step “a” the peroxides are selected from the group comprising, methyl ethyl ketone peroxide, benzoyl peroxide, carbamide peroxide or hydrogen peroxide.

In step “b” the thickeners are selected from the group comprising natrosol, xanthan gum, hydroxymethylcellulose, carbomer, carbopol or combinations thereof, or toothpaste or pure glycerin.

The teeth whitening gel can be used to prepare formulations for teeth whitening.

Tooth whiteners containing modified niobium compounds associated with cations comprise nanostructured niobium compounds modified by previous reaction with acids or peroxides and associated with cations, incorporated into toothpastes or polymer matrices, in the ratio of 0.1 to 30% m/m of niobates in relation to the toothpaste in the ratio of 0.5 to 2% m/m of niobates in relation to the polymer.

The toothpaste used may comprise carboxymethyl cellulose, calcium carbonate, sodium lauryl sulfate and excipients.

The polymer matrix can be selected from the group comprising carbopol and hydroxyethylcellulose.

The niobium compounds used can be selected from the group comprising niobium oxide, niobium pentoxide, niobium ammonium oxalate, niobium chloride and niobium phosphate.

The acids used can be selected from the group comprising oxalic acid, phosphoric acid or hydrofluoric acid, with a concentration of 1 and 70% v/v, and the peroxides can be selected from the group of hydrogen peroxide or organic peroxide, such as benzoyl peroxide or carbamide peroxide in concentrations from 10 to 50% m/v.

The cations associated with modified niobium compounds can be selected from the group comprising Ca²⁺, K⁺, Fe³⁺, Fe²⁺, Mn²⁺, Co²⁺ and cationic methylene blue.

The tooth whitener comprising toothpaste composed of nanostructured niobium is creamy and is indicated for use in oral brushing, resulting in teeth whitening. The tooth whitener comprising niobium compounds and a polymer matrix is in the form of a gel and is indicated for use in tooth whitening with incidence of radiation in the ultraviolet and visible region, at a wavelength of 200 to 800 nm.

The process for obtaining dental whiteners containing modified niobium compounds association with cations comprises the following steps:
a. Modifying niobium precursors selected from the group comprising niobium oxide, niobium pentoxide, niobium ammonium oxalate and niobium phosphate, alone or in combination, with peroxides or acids in a ratio between 1:10 (peroxide) and 1:20 (acids) of leaching agents in relation to niobium compounds;
b. Adjusting the pH of the solution obtained in “a” to values between 1 and 4;
c. Stirring the solution obtained in “b”, with a speed between 100 and 300 rpm, for a period of 1 to 10 minutes;
d. Leaving the solution to rest for a period of 1 to 12 hours;
e. Centrifuging the solution obtained in “d”, with a speed between 2000 and 3000 rpm, and separating the supernatant comprising modified nanostructured niobium compounds;
f. Precipitating the modified nanostructured niobium compounds obtained in “e” from the addition of a cation solution, consisting of cations selected from the group comprising Ca²⁺, Fe³⁺, Fe²⁺, Mn²⁺, Co²⁺ or cationic methylene blue, at a concentration of 100 to 1000 mg/L, to the supernatant obtained in
g. Lyophilizing the gel obtained in “f”;
h. Dispersing the powder obtained in “g” in toothpaste or polymeric matrix of carbopol or hydroxyethylcellulose in the ratio between 0.1 and 30% m/m of niobate in relation to the toothpaste or in the ratio between 0.5 and 2% of niobate for polymer matrix.

The niobium precursors, used to obtain the niobates, described in step “a” can be selected from the group comprising niobium oxide, niobium pentoxide, ammoniacal niobium oxalate, niobium chloride and niobium phosphate. While the acidified solution used for the chemical treatment of step “a” can be selected from oxalic acid, phosphoric acid or hydrofluoric acid, with a concentration between 1 and 70%, or from oxidizing agents with hydrogen peroxide or organic peroxides, such as benzoyl or carbamide peroxide in concentrations from 10 to 50% w/v.

In step “b”, the pH adjustment of the solution can be carried out by adding solutions selected from the group comprising oxalic acid, phosphoric acid or hydrofluoric acid, at a concentration of 0.1 to 1 mol·L⁻¹.

From the leaching step of the niobium compounds and the pH control, the agglomeration of the species occurs, forming anionic oligomers comprising niobium. In step “f”, niobates are precipitated in the form of nanoparticles by reaction with metallic or organic cations, which can be selected from the group comprising Ca²⁺, Fe³⁺, Fe²⁺, Mn²⁺, Co²⁺ and cationic methylene blue at concentrations between 100 and 1000 mg/l.

The present technology can be better understood through the following non-limiting examples.

### Example 1—Obtaining the Whitening Gel from the Active Compound of Niobium Containing Active Oxygen

The whitening gel is obtained by mixing carbopol with the niobium compound, preferably the oxalate, [Nb(O) (C₂O₄)₃]³, but it can be niobium pentoxide, niobic acid, niobium phosphate or alternatively niobium oxyhydroxide.

The niobium compounds were modified with commercial hydrogen peroxide (35% V/V) so that in the whitening gel the concentration is 3% by mass of the peroxide in relation to niobium and carbopol. Commercial carbopol was added in order to obtain a gel in the proportion of 1% by mass of the niobium compound in relation to the carbopol. It is important to emphasize that the whitening gel is stabilized by the presence of carbopol, which, in addition to providing the consistency of the gel, stabilizes the oxygenated radicals formed. Reactive species (oxygen radicals) are formed according to the following three chemical reactions:

—Nb—OH+H₂O₂→—Nb—O—O—H+H₂O (1)

—Nb—O—O—H→—Nb—O*+*OH (2)

—Nb—O*+H₂O→—Nb—OH+*OH (3)

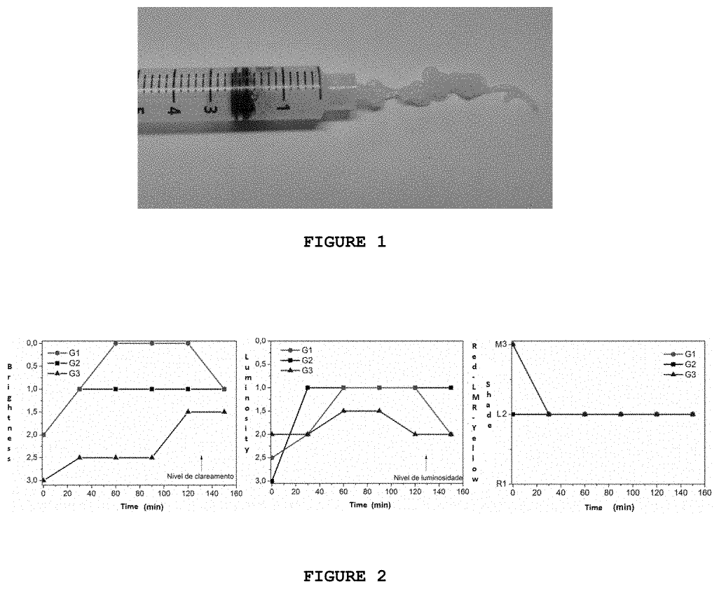

The resulting gel was kept under gentle agitation for 40 min. FIG. 1 shows a photograph of the final gel obtained containing the active oxygen species ready for use as a teeth whitener.

### Example 2—Teeth Whitening Tests in the Presence of Low H₂O₂ Content (2% m/m)

Twelve bovine teeth were selected that were cleaned and preserved in thymol solution (0.1%). The teeth were randomly divided into 6 groups (n=2) and properly identified (Table 1). The initial buccal surface shade for the middle third of each tooth was determined using the VITA Easy Shade spectrophotometer (VITA, Bad Sackingen, Germany). Teeth whitening levels were compared to the VITA 3D Master scale (Vita, Bad Sackingen, Germany). Photographs of bovine teeth were recorded before and after tooth whitening using a Canon T6i camera, macro 100 mm, manual mode, speed 1/160, diaphragm 5.0, ISO 1600, without the use of flash.

TABLE 1
Composition of materials applied to teeth whitening

| Group | Composition |
|---|---|
| G1 | Carbopol, catalyst (niobium compound), |
| | hydrogen peroxide (2%) |
| G2 | Carbopol, catalyst (niobium compound |
| | previously treated with hydrogen peroxide), |
| | hydrogen peroxide (2%). |
| G3 | Commercial product-White Class 6% (H2O2), |
| | FGM. |

The whitening tests were performed using hydrogen peroxide as a bleaching agent and niobium compounds. A commercial product for teeth whitening (White Class 6%, FGM) normally used at home or supervised by the Dental Surgeon was tested as a comparison. For the teeth whitening test, the buccal surface of each specimen was covered by a layer of the mixture containing 4.0 g of Carbopol gel, 1% of the niobium compound and an equivalent amount of 2% of hydrogen peroxide. Applications of the material were carried out at times of 30, 60, 90 and 120 minutes in the absence of light radiation and at the end of each application, the measurement of the color of the teeth was performed with the VITA Easy Shade spectrophotometer. After 120 minutes, the effect of hydration was evaluated in which the teeth were immersed in water for another 30 minutes.

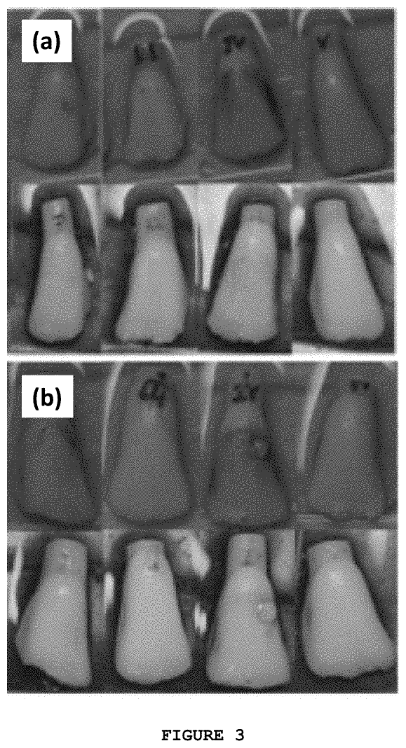

FIG. 2 shows the results of teeth whitening that are represented by the parameters of brightness, intensity of color/luminosity and shade obtained through the VITA 3D MASTER scale.

In addition, the effect of teeth whitening in bovine teeth was qualitatively observed using images obtained by a photographic camera. The images that show the effect of applying the materials G1 and G3 on the teeth are shown in FIG. 3.

The G1 group was the one that presented the best result compared to the others, a color change was detected already from the evaluations with 30 minutes. One point for the brightness parameter and one point for the luminosity parameter. In the evaluation after 60 minutes of application of the teeth whitening gel, there was a gain of one more point for brightness and two for luminosity. This bleaching level stabilized until the last application, totaling 120 minutes (FIG. 2). There was no change for the shade parameter, as expected.

The catalysts with previous chemical treatment (G2) showed lower efficiency in the teeth whitening process when compared to G1. The only recorded evolution of bleaching was only one point in the luminosity parameter. Treatment of the solid with hydrogen peroxide generates reactive oxygen species through the interaction of Bronsted acid sites (Nb—OH) or Nb═O groups, present on the surface of the niobium compound, with H₂O₂. In both materials (G1 and G2) the formation of these species occurs, but the in situ formation of the oxygenated groups, as in G1, potentiates teeth whitening through the efficient oxidation of the dye molecules responsible for the color of the tooth. G3 presented the evolution in whitening in the parameters of brightness and luminosity, 3 and 1 points, respectively. The change in the shade parameter (1 point) was not significant for the assessment of the bleaching level, as can also be seen in FIG. 3.

The results of teeth whitening using the teeth whitening gel of the present invention, in the presence of hydrogen peroxide (2%), are similar to bleaching using the commercial product FGM which has a higher concentration of hydrogen peroxide (6%). The gel that presented the best performance for teeth whitening, G1, was tested in the presence of different concentrations of hydrogen peroxide, among them, 0.5 and 1%. Varying the concentration of the bleaching agent in the materials, it was found that the concentration of 2% (G1) maximized the effect of teeth whitening, being more efficient than the commercial material. Lower concentrations of 0.5 and 1% of H₂O₂ showed less expressive results, around 2 points in the bleaching level and were therefore less effective than the commercial product. The reactive oxygen groups formed in situ depend directly on the amount of hydrogen peroxide added to the material, since low concentrations limit the formation of reactive species and consequently the efficiency of teeth whitening is reduced.

The present invention shows that the gel formed with the niobium compound showed a high ability to remove pigmentation in a shorter application time when compared to the commercial product. For comparison purposes, teeth whitening tests were also performed in the absence of the niobium compound and the results showed that the bleaching action was inefficient. Hydrogen peroxide in low concentrations is not enough to whiten teeth and the presence of the niobium catalyst is essential to potentiate the decomposition of hydrogen peroxide forming reactive oxygen species in situ to act in teeth whitening efficiently.

It should be noted that the present invention employs a lower amount of hydrogen peroxide in the teeth whitening process than commercial products widely used by dentists, with significant potential to reduce the side effects of treatment. This fact is of high clinical relevance, since the indiscriminate use of high concentrations of peroxides and prolonged application times can cause undesirable damage to the dental structure, ranging from increased sensitivity to pulp necrosis or degradation of the enamel crystalline structure.

### Example 3—In Vivo Study of the Effect of Teeth Whitening Gel Containing Niobium on Tooth Enamel and Pulp

In order to evaluate the effect of the teeth whitener on tooth enamel, scanning electron microscopy (SEM) images were obtained. The evaluated teeth were extracted from rats submitted to teeth whitening treatment with teeth whitening gel materials with niobium (G1), commercial (G3) for 45 minutes and with the control group, which consists of teeth that were not submitted to treatment with teeth whitener.

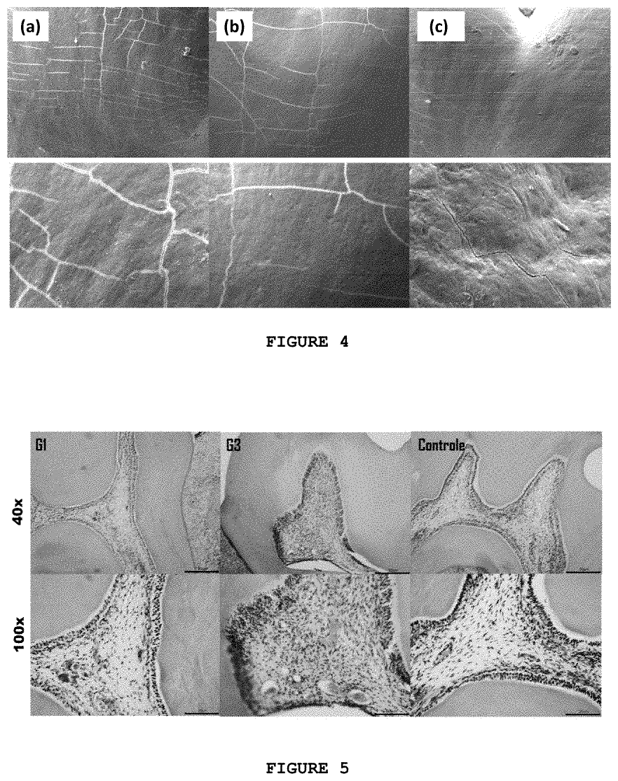

In all the images in FIG. 4 it is possible to note the cracks in the enamel of the teeth, including in the control group. This behavior is normal in rat teeth, which are more sensitive than human teeth, and the effect on human teeth is likely to be less pronounced. The EDS spectra show the presence of the elements Ca, O, P, C, Na, Cl, K and Mg in all teeth. The teeth submitted to application with the teeth whitening gel containing niobium (G1) present a Ca/O and Ca/P ratio of 2.9 and 2.3 respectively. These values are higher than those observed for the teeth submitted to commercial dental bleaching (G3), which presented Ca/O=1.5 and Ca/P=2.1 and also what was observed for the control group, in which Ca/O=1.2 and Ca/P=1.95. These results show that the use of niobium-containing teeth whitening gel as a teeth whitener does not harm the chemical composition of the teeth, and consequently does not cause their demineralization. Therefore, it can be said that the material developed does not have an aggressive effect on tooth enamel.

To evaluate changes in the dental pulp, histological examination was performed on teeth of rats after undergoing teeth whitening using the materials G1 (teeth whitening gel containing niobium), G3 (commercial) and the control group. The work procedure with the animals was carried out, respecting the biosafety standards and recommendations of the Ethics Committee of the Federal University of Minas Gerais (CETEA). From the tests performed with the rats of the Rattus No vergicus group, the images obtained are shown in FIG. 5.

All species of this group exhibited significant changes in the coronal pulp tissue. The images showed a considerable amount of inflammatory cells in the pulp tissue of rats submitted to teeth whitening with the commercial product, containing higher concentration of H₂O₂. In addition, the odontoblast layer is more disorganized for this group, indicating more aggressive effects on the inflammatory process. Inflammation was not significant in rats that were submitted to whitening with the material now developed, exhibiting a similar behavior to the control. In addition, the odontoblastic layer was not altered, showing to be intact, as observed in the control group.

### Example 4—Obtaining Photosensitive Niobates

A dispersion of 2.5 g of niobium pentoxide was obtained with the addition of 50.0 ml of distilled water. Soon after, 4.0 mL of a 35% v/v oxalic acid solution were added. The dispersion was kept under constant stirring at 100 rpm for 10 minutes. After this period, the solution was aged for 12 hours and then centrifuged at a speed of 3,500 rpm. The supernatant, containing negatively charged niobium oligomers, was separated and divided into different aliquots. Solutions containing the following cations separately Ca²⁺, Fe³⁺, Fe²⁺, Mn²⁺ e Co²⁺ were added to each of the supernatant aliquots, at a concentration of 100 mg/L and cationic methylene blue at a concentration of 1,000 mg/L. A gel-like precipitate was formed after the addition of each cation in each aliquot. Afterwards, the gel was lyophilized and, as a result, niobate with powdered cations was obtained. Each cation added to the nanostructured niobate gave rise to a different material and solids were produced with all the mentioned cations.

### Example 5—Dental Whitening Tests with Bovine Teeth Using Visible Light Assisted Niobates

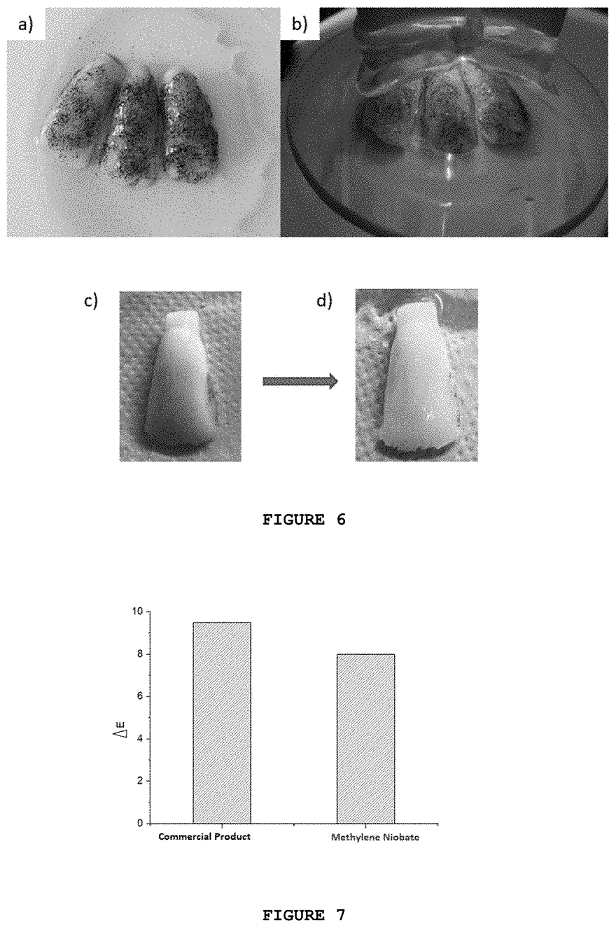

Bovine teeth were cleaned and preserved in thymol solution (0.1%). The initial buccal surface shade for the middle third of each tooth was determined using the VITA Easy Shade spectrophotometer (VITA, Bad Sackingen, Germany). The whitening tests were performed using niobates with methylene, Fe²⁺, Co²⁺ and Mn²⁺ assisted by a source of radiation in the visible region. FIG. 6 shows one of the procedures used in the tooth whitening process. For the tooth whitening test, the buccal surface of each specimen was covered with a layer of niobates dispersed in Carbopol. During the experiments, care was taken to keep the teeth hydrated, thus avoiding the already known optical appearance of greater whiteness when dental dehydration occurs. The mediate loss of water from the subsurface of dental enamel alters the light reflection and refraction indices, giving more temporary whiteness that after hydration is lost. Niobate applications were performed in triplicate and the teeth were in contact with the products for 40 minutes. After the exposure time, the teeth were washed and subjected to a new application of the niobium-based material. Four 40-minute applications were made on each tooth. At the end of each application, the tooth color was measured using the VITA Easy Shade spectrophotometer. FIG. 6c shows the qualitative result obtained from tooth whitening using methylene niobate under visible light.

Teeth whitening levels were compared using the VITA 3D Master scale (Vita, Bad Sackingen, Germany). Photographs of bovine teeth were recorded before and after tooth whitening using a Canon T6i camera, macro 100 mm, manual mode, speed 1/160, diaphragm 5.0, ISO 1600, without the use of flash. The VITA 3D Master Dental Shade Scale is a tool to help determine the shade of natural teeth. Three parameters are evaluated individually to jointly determine the final color of the teeth. They are, 1) brightness: values from 1 to describe teeth with more brightness and with less brightness. Number 1 represents the one with the greatest brightness; 2) color intensity/luminosity: can also be described as color chroma and is measured on a numerical scale (1, 1.5, 2, 2.5 and 3) that represents colors from pale to intense. Number 1 represents the pale; 3) Color Shade: The L-M-R letters indicate different color intensities. The letter L represents yellowish teeth, the letter R represents reddish teeth and the letter M represents the mixture between yellowish and reddish. To measure the results of this study regarding the changes in each of the three parameters described above, scores were assigned. With each modification of each of the three parameters, one (01) point is assigned or withdrawn in case of evolution or regression of whitening. This analysis allows for the determination of the DE, that is, the quantitative difference in whitening obtained.

The AE value represents the color variation of bovine teeth, before and after tooth whitening. The effect of niobates on tooth whitening was compared to the commercial product that uses 35% of peroxide, since the objective is to replace the commercial product that uses hydrogen peroxide as a bleaching agent. FIG. 7 shows that methylene niobate presents efficient teeth whitening results, only slightly lower than the commercial product, which, however, uses a high content of hydrogen peroxide. In addition, methylene niobate presents efficient results in a few tooth whitening sessions and without causing side effects in the patient due to the absence of peroxide. This fact is of high clinical relevance, since the indiscriminate use of high concentrations of peroxides and prolonged application times can cause undesirable damage to the dental structure, ranging from increased sensitivity to pulp necrosis or degradation of the enamel crystalline structure.

Fe, Co or Mn niobates showed similar results. It is important to highlight that tooth whitening using niobates in the presence of radiation proved to be very efficient with few product application sessions, which makes the process faster and less expensive when compared to conventional treatment.

### Example 6—Teeth Whitening Tests with Bovine Teeth Using Niobates as Toothpaste Components

The niobate formed with Ca²⁺ was incorporated, by dispersion using a stirrer, in a toothpaste free from whitening and abrasive compounds (163 mL Deionized water, 3 g of Carboxymethyl cellulose, 225 g of Calcium carbonate, 5 g of Sodium Lauryl Sulfate, 0.5 g of Nipagim, 0.5 g of Saccharin, Sorbitol) prepared especially for the experiment.

For the whitening test, 6 bovine teeth were used in each experimental group (n=6). Fourteen 2-minute brushes were performed. At every one minute the tooth was washed and more paste was added to the brush. Each specimen was brushed for 28 minutes.

The color evaluations were performed in triplicate and in two stages: Initial time T(0) and Final time T(f) were measured by Vita Vita Easyshade 5.0 Visible Reflectance spectrophotometer (VITA Zahnfabrik, Bad Sackingen, Baden-Württemberg, Germany), calibrated according to the manufacturer at each reading.

Teeth whitening levels were compared using the VITA 3D Master scale (Vita, Bad Sackingen, Germany). Photographs of bovine teeth were recorded before and after tooth whitening using a Canon T6i camera, macro 100 mm, manual mode, speed 1/160, diaphragm 5.0, ISO 1600, without the use of flash. The VITA 3D Master Dental Shade Scale is a tool to help determine the shade of natural teeth. Three parameters are evaluated individually to jointly determine the final color of the teeth. They are, 1) brightness: values from 1 to describe teeth with more brightness and with less brightness. Number 1 represents the one with the greatest brightness; 2) color intensity/luminosity: can also be described as color chroma and is measured on a numerical scale (1, 1.5, 2, 2.5 and 3) that represents colors from pale to intense. Number 1 represents the pale; 3) Color Shade: The L-M-R letters indicate different color intensities. The letter L represents yellowish teeth, the letter R represents reddish teeth and the letter M represents the mixture between yellowish and reddish. To measure the results of this study regarding the changes in each of the three parameters described above, scores were assigned. With each modification of each of the three parameters, one (01) point is assigned or withdrawn in case of evolution or regression of whitening. This analysis allows for the determination of the DE, that is, the quantitative difference in whitening obtained.

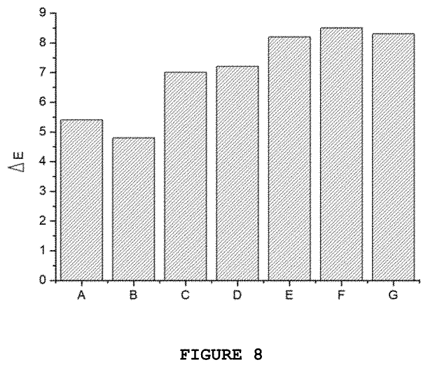

FIG. 8 presents comparisons between the quantified AE for different samples of niobate toothpaste and commercial toothpaste containing 2% of hydrogen peroxide. Sample A corresponds to the commercial product presenting ΔE=5.3. Sample B corresponds to the niobium oxide used directly, without the transformation into niobates described in the present application. Sample B showed low bleaching power (DE=4.8), the effect being due to the abrasive effect. Samples C, D, and E correspond to niobate solidified without addition of cation, niobate solidified with Ca²⁺ and niobate solidified with e³⁺, all in the ratio of 3% m/m of the material in the toothpaste. Samples F and G correspond to niobates obtained from commercial niobium phosphate in toothpaste, in the proportion of 1% and 2%, respectively. Thus, the compounds obtained to be used as whiteners, samples C, D, E, F and G, whose DE values are 6.9; 7.1; 8.1; 8.7 and 8.6, respectively, show whitening results above the commercial compound that claims this property.

The present invention is relevant for the development of materials for dental application based on niobium compounds to produce a toothpaste/paste using commercial or synthetic materials, as an alternative to the materials currently used, mainly in dental offices. Furthermore, because this invention employs a photosensitive compound that, in the presence of light, it generates reactive species with a high capacity to oxidize molecules impregnated in the teeth that give it color, favoring tooth whitening. It is important to highlight that the whitening process occurs without the need to use peroxides, eliminating the side effects of the treatment currently used.

1. A TEETH WHITENER comprising nanostructured niobium compounds modified by reaction with acids or peroxides and thickeners, in the ratio between 1 and 50% by mass of the niobium compound in relation to the thickener.

2. THE TEETH WHITENER according to claim 1, wherein the peroxides are selected from the group comprising methyl ethyl ketone peroxide, benzoyl peroxide, carbamide peroxide, or hydrogen peroxide, with a purity between 30 and 70%, in concentrations between 1, 0 and 10.0% m/m of peroxide in relation to the total mass of the gel (thickener and niobium compound).

3. THE TEETH WHITENER according to claim 1, wherein the acids are oxalic acid, phosphoric acid or hydrofluoric acid, with a concentration of 1 to 70% v/v, and wherein the peroxides are hydrogen peroxide, benzoyl peroxide or carbamide peroxide, with a concentration of 1 to 50% w/v.

4. THE TEETH WHITENER according to claim 1, wherein the niobium compounds are selected from the group consisting of niobium oxide, niobium pentoxide, niobium ammonium oxalate and niobium phosphate, wherein the niobium sources being able to be isolated or in combination, and associated with cations, incorporated in toothpastes or polymer matrices, in the ratio of 0.1 to 30% m/m of niobate in relation to the paste, or in the ratio of 0.5 to 2% m/m of niobate in relation to the polymer.

5. THE TEETH WHITENER according to claim 4, wherein the toothpaste comprises carboxymethyl cellulose, calcium carbonate, sodium lauryl sulfate and excipients.

6. THE TEETH WHITENER according to claim 4, wherein the cations are selected from the group consisting of Ca²⁺, K⁺, Fe³⁺, Fe²⁺, Mn²⁺, Co²⁺ and cationic methylene blue.

7. THE TEETH WHITENER according to claim 1, further comprising nanostructured niobium compounds modified by reaction with peroxides and the thickener selected from the group consisting of natrosol, xanthan gum, hydroxymethylcellulose, carbomer, carbopol and combinations thereof, or toothpaste or pure glycerin.

8. THE TEETH WHITENER according to claim 7, wherein the niobium compounds are selected from the group consisting of niobium phosphates, niobium oxides, acetates, chlorides, niobium filter cake, niobia, and the oxalate anion ([Nb(O) (C₂O₄)₃]³⁻).

9. A METHOD FOR PREPARING THE TEETH WHITENER according to claim 7, comprising the following steps:
a) modifying the niobium compounds with hydrogen peroxide of purity between 30 and 70%, using peroxide concentrations between 1.0 and 10.0% m/m in relation to the total mass (thickener and niobium compound);
b) adding the thickener in the ratio of 1 to 10% by mass of the modified niobium compound obtained in step “a” in relation to the thickener;
c) mixing the composition obtained in “b” under gentle agitation between 50 and 1000 rpm for a time interval between 10 and 60 min, at room temperature.

10. THE METHOD OF PREPARING THE TEETH WHITENER according to claim 9, wherein in step “a”, the niobium compounds are selected from the group consisting of niobium phosphates, niobium oxides, acetates, chlorides, niobium filter cake, niobia, and the oxalate anion ([Nb(O) (C₂O₄)₃]³⁻).

11. THE METHOD FOR PREPARING THE TEETH WHITENER according to claim 9, wherein in step “a”, the peroxides are selected from the group consisting of methyl ethyl ketone peroxide, benzoyl peroxide, carbamide peroxide and hydrogen peroxide.

12. THE METHOD FOR PREPARING THE TEETH WHITENER according to claim 9, wherein in step “b”, the thickener is selected from the group consisting of natrosol, xanthan gum, hydroxymethylcellulose, carbomer, carbopol and combinations thereof, or toothpaste or pure glycerin.

13. THE METHOD OF OBTAINING TEETH WHITENER according to claim 4, comprising the following steps:
a) modifying niobium precursors selected from the group comprising niobium oxide, niobium pentoxide, niobium ammonium oxalate and niobium phosphate, alone or in combination, with peroxides or acids in a ratio between 1:10 (peroxide) and 1:20 (acids) of leaching agents in relation to niobium compounds;
b) adjusting the pH of the solution obtained in step a) to values between 1 and 4, by adding an acid solution selected from the group comprising oxalic acid, phosphoric acid or hydrofluoric acid in a concentration between 0.1 and 1 mol/L;
C) stirring the solution obtained in step b), with a speed between 100 and 300 rpm, for 10 minutes;
d) leaving the solution to rest for a period of time between 1 and 12 hours;
e) the solution obtained in step d), with a speed between 2000 and 3000 rpm, separating the supernatant comprising modified nanostructured niobium compounds;
f) precipitating the modified nanostructured niobium compounds obtained in step e) from the addition of cation solution to the supernatant obtained in step e);
g) lyophilizing the gel obtained in step f);
h) dispersing the powder obtained in step g) in toothpaste or polymer matrix in the ratio between 0.1 and 30% m/m of niobate in relation to the toothpaste or in the ratio between 0.5 and 2% m/m of niobium compounds for polymer matrix.

14. THE METHOD OF OBTAINING TEETH WHITENER according to claim 13, wherein in step a), the acidified solution consists of distilled water and a compound selected from the group comprising oxalic acid, phosphoric acid or hydrofluoric acid, in a concentration of 1 to 70% v/v and the peroxides are selected from hydrogen peroxide, benzoyl peroxide or carbamide peroxide, with concentrations from 1 to 50% w/v.

15. A FORMULATION FOR TEETH WHITENING comprising the teeth whitener according to claim 1.

16. A TOOTHPASTE FOR ORAL BRUSHING AND TEETH WHITENING comprising the formulation of claim 15.

17. A METHOD FOR PRODUCING A POLYMER MATRIX GEL comprising the formulation according to claim 15, wherein the method for the producing the polymer matrix gels for tooth whitening is carried out by activating from the incidence of radiation in the visible and ultraviolet region, at a wavelength from 200 to 800 nm.
